(19) United States
(12) Patent Application Publication
(10) Pub. No.: US 2022/0227728 A1
(43) Pub. Date: Jul. 21, 2022

(54) Microtubule-associated protein Tau imaging compounds for Alzheimer's disease and precursors thereof

(71) Applicants: MING-HSIN LI, TAOYUAN CITY (TW); KAI-HUNG CHENG, TAOYUAN (TW); SHENG-NAN LO, TAOYUAN (TW); SHIH-WEI LO, TAOYUAN (TW); YUAN-RUEI HUANG, TAOYUAN (TW); JENN-TZONG CHEN, TAOYUAN (TW)

(72) Inventors: MING-HSIN LI, TAOYUAN CITY (TW); KAI-HUNG CHENG, TAOYUAN (TW); SHENG-NAN LO, TAOYUAN (TW); SHIH-WEI LO, TAOYUAN (TW); YUAN-RUEI HUANG, TAOYUAN (TW); JENN-TZONG CHEN, TAOYUAN (TW)

(21) Appl. No.: 17/152,344

(22) Filed: Jan. 19, 2021

(51) Int. Cl. C07D 401/04; A61K 51/04

(52) U.S. Cl. CPC C07D 401/04; C07B 2200/05; A61K 51/0455

(57) ABSTRACT

The present invention discloses a series of nuclear medicine tracers that are combined with brain microtubule-associated protein Tau targeting compounds to produce a group of compounds of nuclear medicine that can be utilized for imaging of microtubule-associated protein Tau. When the positrons released by the decay encounter the electrons of the cells in the sample, utilizing the positron decay characteristics of fluorine-18 or iodine-124 isotope to generate mutual destruction reactions, a pair of opposite gamma rays is formed which are imaged by positron emission tomography. The compounds can be applied for the in vivo detection of microtubule-associated protein Tau deposits in the brain. The invention provides a strategy for diagnosis of Alzheimer's disease and a method to measure the efficacy of therapeutic drugs targeting microtubule-associated protein Tau.

### BACKGROUND OF THE INVENTION

### 1. Field of the Invention

The present invention relates to an imaging compound and a precursor of microtubule-associated protein Tau thereof, in particular, relates to labeling radionuclides and producing a series of new nuclei medical tracer for Alzheimer's disease.

### 2. Description of Related Art

Alzheimer's Disease (AD) is an irreversible and persistent neurodegenerative disease. The main symptoms are labyrinth, memory decline, cognitive dysfunction, emotional instability, and behavioral changes. The two most important pathological features are abnormal plaques and neurofibrillary tangle in the brain. Plaques are mainly formed by the accumulation of beta amyloid (Aβ), and the neurofibrillary tangle is mainly caused by the hyperphosphorylation of the microtubule-associated protein Tau, which causes the microtubules to be twisted and deformed and accumulated in the cells.

The main function of the microtubule-associated protein Tau is to bind to microtubules and stabilize brain nerve cells. In recent years, studies have also found that changes in the amount of abnormal microtubule-associated protein Tau are positively correlated with Alzheimer's disease, including increased levels of microtubule-associated protein Tau in plasma are related to cognitive decline and control in the brain atrophy of the memory hippocampal gyms is related to the increase in the concentration of the microtubule-associated protein Tau in the cerebrospinal fluid. At the same time, abnormal microtubule-associated protein Tau can also cause rare brain diseases, such as frontotemporal dementia (FTD) and progressive supranuclear palsy (PSP). Therefore, starting from the microtubule-associated protein Tau, there is an opportunity to find a key in diagnosis and treatment of Alzheimer's disease.

Dementia is a disease phenomenon, not a normal aging. The symptoms are not only memory loss, but also affect other cognitive functions, including language, space, calculation, judgment, abstract thinking, attention and other aspects of functional degradation, which are severe enough to interfere with daily life. The current drugs for dementia have no way to stop or restore damaged brain cells, but they may improve the patient's symptoms or delay the progression of the disease. At the same time, because the causes of dementia have not been clearly understood, clinical or preclinical research has not yet found the most suitable strategy for early diagnosis of this disease.

Dementia is mainly divided into degenerative dementia, vascular dementia, and other causes of cognitive dysfunction. Among them, Alzheimer disease is the most common, accounting for about 60% of cases of dementia Alzheimer's disease is an irreversible and progressive neurodegenerative disease of the brain.

Research in recent years has found that changes in the amount of abnormal microtubule-associated protein Tau are positively correlated with Alzheimer's disease. In May 2019, the Johns Hopkins University research team even found that using the microtubule-associated protein Tau as a biomarker can detect Alzheimer's disease 34 years before the onset.

In addition, abnormalities in the microtubule-associated protein Tau can also cause rare brain diseases, such as frontotemporal dementia (FTD), progressive supranuclear palsy (PSP), corticobasal degeneration (CBD), chronic traumatic encephalopathy (CTE) and Pick's disease, etc. Therefore, starting from the microtubule-associated protein Tau, there is an opportunity to find a key in diagnosis and treatment of Alzheimer's disease.

The microtubule-associated protein Tau positron contrast agent is a non-invasive way to identify the number and distribution of the microtubule-associated protein Tau fibrillation in the brain to find possible patients. At the same time, it also allows drug researchers to evaluate the biochemical changes or metabolic effects of candidate drugs in the body for a long time to identify potential therapeutic drugs for microtubule-associated protein Tau.

The development of PET contrast agents that use the microtubule-associated protein Tau as a biological indicator is in full swing. These drugs include the first generation of contrast agents: ¹⁸T-FDDNP, 11C-PBB3, ¹⁸F-THK5351, ¹⁸-THK5105, ¹⁸F-THK523, ¹⁸F-THK5117, ¹⁸F-AV1451, Lansoprazole series and ¹⁸F-T808, etc.; second-generation contrast agents: ¹⁸F-MK6240, ¹⁸F-GTP1, ¹⁸F-PM-PBB3, ¹⁸F-AM-PBB3, ¹⁸F-PI-2620, ¹⁸F-JNJ311 and ¹⁸F-RO6958948. Among them, the drugs that have progressed to clinical phase III trials are only ¹⁸F-AV1451, and the drugs that have progressed to clinical phase II trials are ¹⁸F-MK6240, ¹⁸F-PM-PBB3, ¹⁸F-THK5351, ¹⁸F-PI-2620 and ¹⁸F-GTP1, and the drugs in clinical phase 1 trials include ¹⁸F-FDDNP, ¹⁸F-RO6958948 and ¹⁸F-T808.

As there is no approved drug available now for use with the microtubule-associated protein Tau positron contrast agent, and there are many international pharmaceutical companies engaged in the development in searching for potential effective drugs.

The current dementia diagnosis process will first clarify the medical history and necessary physical, neurological, and mental status examinations to rule out delirium, depression, and drugs that cause dementia. If it is still suspected to be dementia or the prodromal stage of dementia after the exclusion, a standard inspection process including cognitive function testing, such as MMSE (Mini-Mental State Examination), CASI (Cognitive Abilities Screening Instrument), CERAD (Consortium to Establish a Registry for Alzheimer's Disease), laboratory tests, including complete blood count, biochemical, vitamin B12, Folic acid, syphilis serum, thyroxine, and thyroid secretion promoting hormone, and head brain tomography and magnetic resonance imaging. However, according to the above procedure, most patients are already in the middle or late stage when they are examined or diagnosed. In particular, the diagnosis of Alzheimer's disease has only 80% sensitivity and 70% specificity, and the accuracy of clinical diagnosis is only up to a moderate level.

Therefore, positron radiography, which can diagnose early and has high accuracy, is considered a powerful tool for the current diagnosis of dementia. Several positron contrast agents based on the Amyloid hypothesis have been approved by the U.S. Food and Drug Administration (FDA) after 2012, including ¹⁸F-Florbetapir, ¹⁸F-Flutemetamol and ¹⁸F-Florbetaben; However, drugs based on the Tau hypothesis of the microtubule-associated protein have not yet been marketed.

In 2018, the National Dementia Association (NIA-AA) officially included the microtubule-associated protein Tau into the benchmark, including the content of microtubule-associated protein Tau in cerebrospinal fluid (CSF) and the photo of the microtubule-associated protein Tau positron. However, the acquisition of cerebrospinal fluid samples is by an invasive method, which has the risk of infection and patients are prone to discomfort. Therefore, the application potential of the microtubule-associated protein Tau positron imaging is worth further researching, and many international pharmaceutical companies have already invested in its development.

The current development of the first-generation microtubule-associated protein Tau contrast agent may deviate from the target absorption (off-target). For example, the first-generation contrast agent such as ¹¹C-PBB3 and ¹⁸F-AM-PBB3 is compared with the second-generation contrast agent ¹⁸F-PI-2620 and found that the absorption in the venous sinus is significantly higher. Other first-generation contrast agents such as ¹⁸F-THK-5351, ¹⁸F-THK-5317 or ¹⁸F-AV-1451 has indicated higher absorption in the striatum area than the second-generation contrast agents ¹⁸F-PI-2620 and ¹⁸F-MK-6240 in normal human brain imaging.

### SUMMARY OF THE INVENTION

The primary purpose of the present invention is to label the four compounds, including INER-TAU-R3 and its analogues INER-TAU-R2, INER-TAU-R1, and INER-TAU-I with radionuclides F-18 or iodine, to produce a series of new nuclear medicine tracers, for example, ¹⁸F-INER-TAU-R3, ¹⁸F-INER-TAU-R2, ¹⁸F-INER-TAU-R1, which can be combined with brain microtubules-associated protein Tau to produce a novel compound group that focuses on the nuclear medicine imaging for the microtubule-related protein Tau.

Another object of the present invention is to provide a novel compound for diagnosing microtubule-associated protein Tau for neurodegenerative diseases, which utilizes the positron decay characteristics of fluorine-18 or iodine-124 isotopes. When the positrons released by the decay encounter electrons in cells that produce an annihilation reaction, forming a pair of opposite directions of 511 keV gamma rays, and images can be obtained to study the microtubule-related protein Tau deposited in the brain by positron emission tomography (PET) to enable the diagnosis of Alzheimer's disease and other neurodegenerative diseases characterized by the pathology of microtubule-associated protein Tau, and to provide methods for measuring the efficacy of therapeutic drugs targeting microtubule-associated Tau protein.

Another objective of the present invention is to disclose the structure of the novel compound having the overall pharmacophore structure similar to the second-generation microtubule-associated protein Tau contrast agent, but the carbon chain of the ¹⁸F source labeled is elongated, thereby keeping away from the pharmacophore, so it is not easy to affect the activity. At the same time, the removal of a nitrogen from the benzene ring structure of the pharmacophore not only reduces the synthesis difficulty, but also reduces the synthesis cost.

In addition, the physicochemical properties of the compounds disclosed in the present invention are in line with current common physicochemical properties of central nerve drugs, including the logarithmic value of the lipid-water partition coefficient (log P) is between 2 and 5, the molecular weight is ≤450, and the topological polar surface area (TPSA)<90 Å², the number of hydrogen bond donors is <3, the number of hydrogen bond acceptors is <7, and the number of rotatable bonds is between 0-8. At the same time, its fat solubility is greater and the topological polarity surface area is smaller than the second-generation microtubule-associated protein Tau contrast agent referred to, so the blood-brain barrier may have better communication properties. In the current animal experiments, positron computed tomography (PET) methods have also confirmed that this compound does enter the brain and binds to the microtubule-associated protein Tau in the brain.

Another object of the present invention is to disclose a Tau compound precursor of a microtubule-associated protein for diagnosing neurodegenerative diseases. The leaving group position for nucleophilic substitution reaction is designed into an OTs structure. The precursor with the OTs structure is stable and easy to store. In addition, it is easier to label the fluorine-18 isotope, and the yield is also higher. The actual labeling reaction process only needs 15 minutes at 95° C.

Another object of the present invention is to provide labeling precursors, standards, and tracers for targeted microtubule-associated protein Tau compound INER-TAU-R3 and its derivatives, which are divided into two major sub-items: the first sub-item is INER-TAU-I1 and INER-TAU-I2 compound labeling precursors and tracers, of which the labeling method is to replace the I position of the precursor INER-TAU-I1, INER-TAU-I2 through isotope exchange with iodine isotope (¹²³I, ¹²⁴I, ¹²⁵I, or ¹³¹I). The second sub-item is INER-TAU-R3, INER-TAU-R2, INER-TAU-R1 and other three compound labeling precursors, standards and tracers, of which the labeling method is to carry out nucleophilic substitution reaction and replace OTs position with fluorine-18 isotope at OTs position of rear leaving group of the precursor INER-TAU-R3-OTs, INER-TAU-R2-OTs, INER-TAU-R1-OTs, and produce a series of new nuclear medicine tracer.

### BRIEF DESCRIPTION OF THE DRAWINGS

FIG. 1 is a radio-TLC (radioactive thin layer chromatography) analysis diagram of the microtubule-associated protein Tau compound 29 of the present invention, in which the thin layer sheet Silica gel 60 F254 (Merck brand) is used for the radio-TLC analysis, the mobile phase is dichloromethane:methanol, the ratio is 80:20. The sample of compound 29 was spotted on the thin layer chromatographic sheet and use the mobile phase to elute, and the chromatographic sheet after elution was then analyzed with a radio-TLC imaging scanner (Brand BIOSCAN), and the mobile ratio of compound 29 (Ratio of flow, Rf) is about 0.8.

FIG. 2 is a radio-HPLC (radio-High Performance Liquid Chromatography) analysis diagram of the microtubule-associated protein Tau compound 29 of the present invention, in which the radio-HPLC (Waters brand) uses the column XSelect HSS T3 3.5 μm, and the mobile phase is a gradient from 60% acetonitrile plus 0.1% trifluoroacetic acid to 90% acetonitrile plus 0.1% trifluoroacetic acid in 30 minutes at a flow rate of 0.8 ml per minute, using a radioactive detector (BIOSCAN brand). The results of the analysis of the compound 29 sample by radio-HPLC showed that the retention time of its main peak was at about 22.503 minutes.

FIG. 3 is an image of the microtubule-associated protein Tau compound 29 of the present invention in P301L gene transgenic mice and control mice using positron emission tomography (nano PET/CT) analysis, in which P301L gene transgenic mice (source: United States JACKSON Research Center) refers to the mutation of proline to leucine at codon 301 of the encoded microtubule-associated protein Tau. These types of mutations can accelerate the formation of neurofibrillary tangles. P301L gene transgenic mice and control mice were injected through the tail vein with 0.46 millicuries (mCi) of compound 29 of the present invention, and after 30 minutes of distribution, radiography was performed on the mice using a positron emission tomography scanner (BIOSCAN). The image results showed that the drug accumulation of P301L in the brain of gene transgenic mice (A) was significantly greater than that of control mice (B).

### DETAILED DESCRIPTION OF THE PREFERRED EMBODIMENTS

### Embodiment 1

### Synthesis of INER-TAU-I1 Series Compounds

Synthesis of precursor 6-iodo-3-(2H-isoindol-2-yl) isoquinoline, comprising:

1. Adding the compound 1, 6-fluoro-3-(2H-isoindol-2-yl) isoquinoline 9.5 mmole, to a saturated solution of ammonia dissolved in methanol, stirring and being reacted for 5 days at 120° C. to obtain compound 2, 3-(2H-isoindol-2-yl) isoquinoline-6-amine: MS (mass spectrometry) (m/z) 260.1 [M+H]⁺.

2. Suspending compound 2 in water, adding concentrated sulfuric acid at room temperature and heating to 70-80° C. to obtain a clear solution, and the mixed solution was cooled to 0° C. and sodium nitrate was added and reacted at 0° C. for 15 minutes, then potassium iodide, in which molar ratio of compound 2:sodium nitrate:potassium iodide=1:1.98:1.98, was added and reacted for 5 minutes to obtain compound 3, 6 iodo-3-(2H-isoindol-2-yl) isoquinoline: MS (m/z) 371.0 [M+H]⁺, as shown:

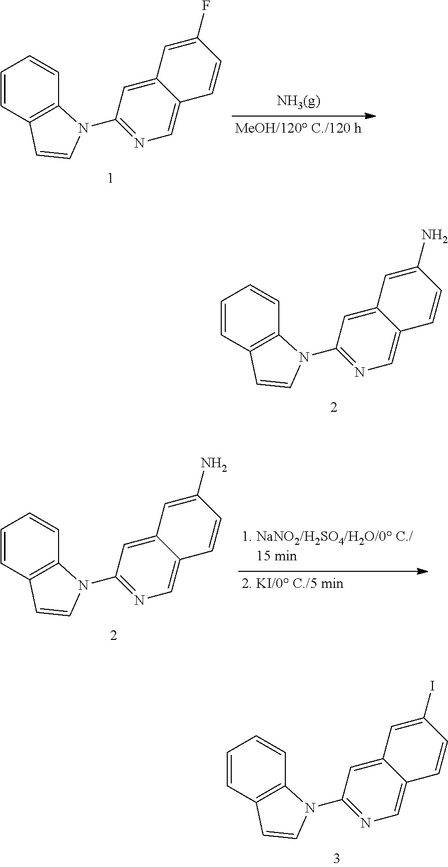 [SMILES: Fc1ccc2cnc(-n3ccc4ccccc43)cc2c1 ; Nc1ccc2cnc(-n3ccc4ccccc43)cc2c1 ; Nc1ccc2cnc(-n3ccc4ccccc43)cc2c1 ; Ic1ccc2cnc(-n3ccc4ccccc43)cc2c1]

Synthetic of tracer [¹²³I]6-iodo-3-(2H-isoindol-2-yl) isoquinoline:

Adding sodium iodide ([¹²³I]NaI) in 0.01N NaOH and Na₂S₂O₅ to the reaction flask and is dried with nitrogen at 100° C., adding compound 3 dissolved in acetic acid and cuprous sulfate solution and reaction at 180° C. for 10 minutes, and after being re-dissolved in ethanol the compound 4 is obtained, as shown:

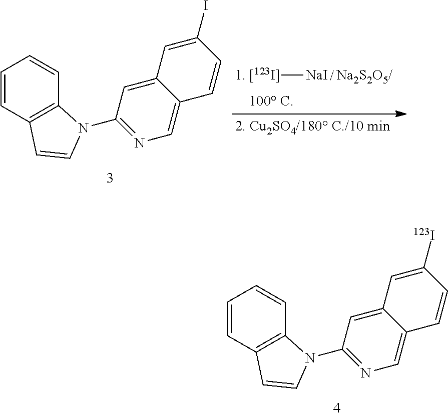

Synthesis of tracer [¹²⁴I]6-iodo-3-(2H-isoindol-2-yl) isoquinoline:

Sodium iodide ([¹²⁴I]NaI) in 0.01N NaOH and Na₂S₂O₅ was added to the reaction flask and dried with nitrogen at 100° C., then compound 3 dissolved in acetic acid and cuprous sulfate solution were added and reacted at 180° C. for 10 minutes, and after being re-dissolved in ethanol and purification the compound 5 is obtained:

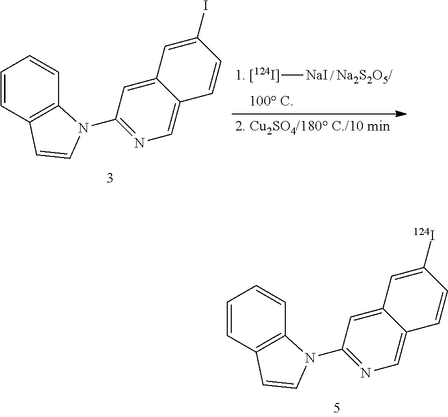

Synthesis of tracer [¹²⁵I]6-iodo-3-(2H-isoindol-2-yl) isoquinoline:

Sodium iodide ([¹²⁵I]NaI) in 0.01N NaOH and Na₂S₂O₅ was added to the reaction flask and dried with nitrogen at 100° C., then compound 3 dissolved in acetic acid and cuprous sulfate solution were added and reacted at 180° C. for 10 minutes, and after being re-dissolved in ethanol and purification the compound 6 is obtained:

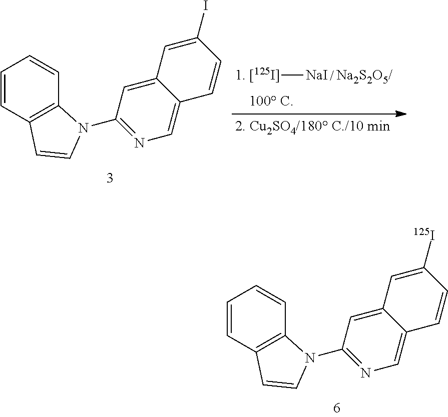

Synthesis of tracer [¹³¹I]6-iodo-3-(2H-isoindol-2-yl) isoquinoline:

Sodium iodide ([¹³¹I]NaI) in 0.01N NaOH and Na₂S₂O₅ was added to the reaction flask and dried with nitrogen at 100, then compound 3 dissolved in acetic acid and cuprous sulfate solution were added and reacted at 180° C. for 10 minutes, and after being re-dissolved in ethanol and purification the compound 7 is obtained:

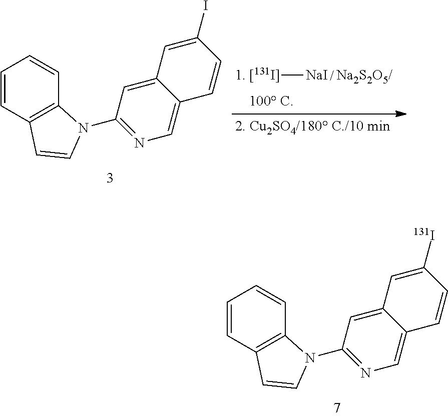

### Embodiment 2

### Synthesis of INER-TAU-I2 Series Compounds

Synthesis of precursor 5-iodo-3-(2H-isoindol-2-yl)isoquinoline, comprising:

1. 3-chloroisoquinolin-5-amine compound 8 is dissolved in dichloromethane, and triethylamine and di-tert-butyl dicarbonate were added, in which molar ratio of compound 8:triethylamine:di-tert-butyl dicarbonate=1:2:1.2, the mixture was reacted at room temperature for 3 hours, and then concentrated and purified under reduced pressure to obtain compound 9, tert-butyl (3-chloroisoquinolin-5-yl) carbamate: MS (m/z) 279.1 [M+H]⁺.

2.3H-Indole and compound 9 were dissolved in tetrahydrofuran and added sodium 2-methylpropan-2-olate and t-BuXPhos palladium(II) biphenyl-2-amine mesylate were added, in which molar ratio of 3H-Indole:compound 9:sodium 2 -methylpropan-2-olate: t-BuXPhos palladium(II) biphenyl-2-amine mesylate=1.5:1:2:0.1, the mixed solution was filled with nitrogen and reacted at 50° C. for 16 hours, purified to obtain compound 10, tert-butyl 3-(2H-isoindol-2-yl)isoquinolin-5-ylcarbamate: MS (m/z) 360.2 [M+H]⁺.

3. Compound 10 was dissolved in dichloromethane solution, and trifluoroacetic acid, in which molar ratio of compound 10:trifluoroacetic acid=1:10, was added at room temperature and reacted for 1 hour, and then was extracted and purified to obtain compound 11, 3-(2H-isoindol-) 2-yl) isoquinolin-5-amine:MS (m/z) 260.1 [M+H]⁺.

4. Compound 11 was dissolved in 10% sulfuric acid solution, and sodium nitrate was added to react at ° C. for 20 minutes, then potassium iodide was added, in which compound 11:sodium nitrate:potassium iodide=1:2:2, and then extracted and purified for 10 minutes to obtain compound 12, 5-iodo-3-(2H-isoindol-2-yl)isoquinoline: MS (m/z) 371.0 [M+H]⁺. as shown:

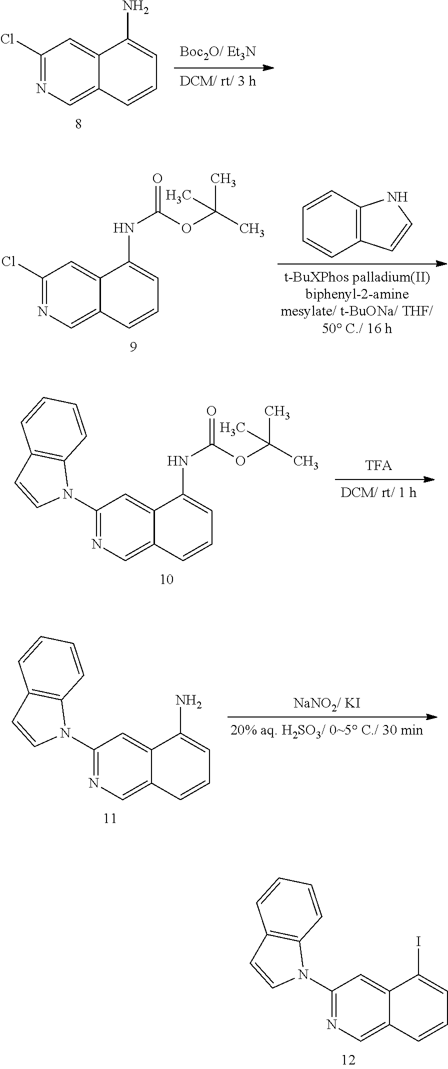 [SMILES: Nc1cccc2cnc(Cl)cc12 ; CC(C)(C)OC(=O)Nc1cccc2cnc(Cl)cc12 ; c1ccc2[nH]ccc2c1 ; CC(C)(C)OC(=O)Nc1cccc2cnc(-n3ccc4ccccc43)cc12 ; Nc1cccc2cnc(-n3ccc4ccccc43)cc12 ; Ic1cccc2cnc(-n3ccc4ccccc43)cc12]

Synthesis of tracer [¹²³I]5-iodo-3-(2H-isoindol-2-yl)isoquinoline:

Sodium iodide ([¹²³I]NaI) in 0.01N NaOH and Na₂S₂O₅ were added to the reaction flask and dried with nitrogen at 100° C., and compound 12 dissolved in acetic acid and cuprous sulfate solution were added and reacted at 180° C. for 10 minutes, and after being re-dissolved in ethanol and purification the compound 13 is obtained:

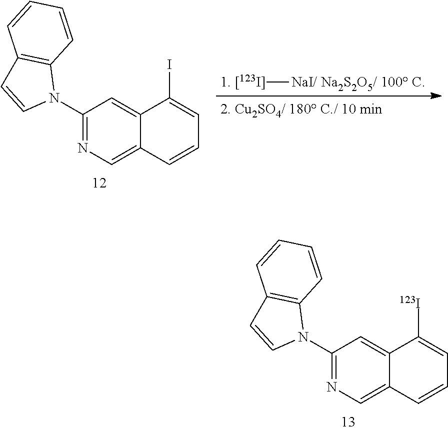

Synthesis of tracer [¹²⁴I]5-iodo-3-(2H-isoindol-2-yl)isoquinoline:

Sodium iodide ([124I]NaI) in 0.01N NaOH and Na2S2O5 were added to the reaction flask and dried with nitrogen at 100° C., and compound 12 dissolved in acetic acid and cuprous sulfate solution were added and reacted at 180° C. for 10 minutes, and after being re-dissolved in ethanol and purification the compound 14 is obtained:

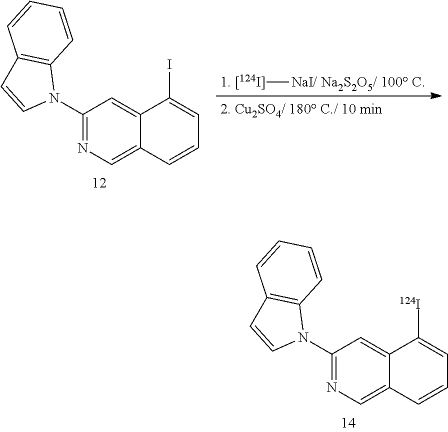

Synthesis of tracer [¹²⁵I]5-iodo-3-(2H-isoindol-2-yl)isoquinoline:

Sodium iodide ([¹²⁵I]NaI) in 0.01N NaOH and Na2S2O5 were added to the reaction flask and dried with nitrogen at 100° C., and compound 12 dissolved in acetic acid and cuprous sulfate solution were added and reacted at 180° C. for 10 minutes, and after being re-dissolved in ethanol and purification the compound 15 is obtained:

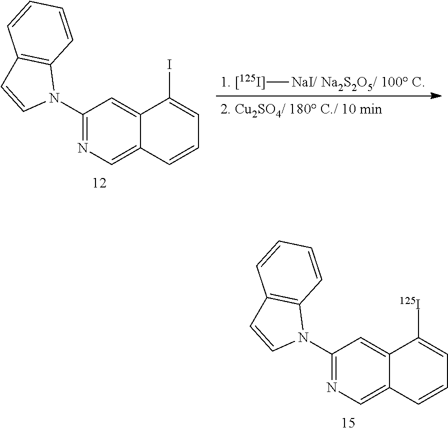

Synthesis of tracer [¹³¹I]5-iodo-3-(2H-isoindol-2-yl)isoquinoline:

Sodium iodide ([¹³¹I]NaI) in 0.01N NaOH and Na₂S₂O₅ were added to the reaction flask and dried with nitrogen at 100° C., and compound 12 dissolved in acetic acid and cuprous sulfate solution were added and reacted at 180° C. for 10 minutes, and after being re-dissolved in ethanol and purification the compound 16 is obtained:

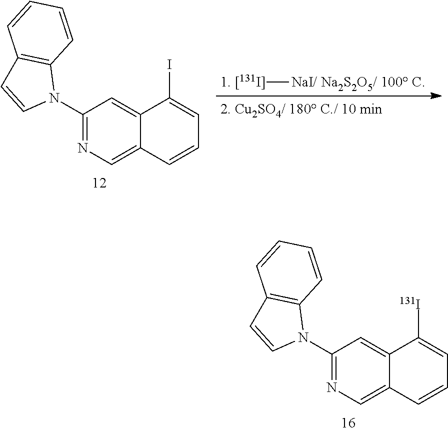

### Embodiment 3

### Synthesis of INER-TAU-R1 Series Compounds

Synthesis of precursor 7-(2-p-toluenesulfonyloxymethoxy)-3-(2H-isoindol-2-yl) isoquinoline, comprising:

1. 1,3-dichloro-7-methoxyisoquinoline compound 17 is dissolved in a mixed solution of acetic acid and 45% iodine hydride, and red phosphorous is added at room temperature, and the mixed solution is reacted at 100° C. for 4 hours, then cooled to room temperature and purified to obtain compound 18, 3-chloro-7-methoxyisoquinoline: MS (m/z) 194.0 [M+H]⁺.

2. 3H-Indole and compound 18 were dissolved in tetrahydrofuran and added sodium 2-methylpropan-2-olate and t-BuXPhos palladium(II) biphenyl-2-amine mesylate at room temperature, in which molar ratio of 3H-Indole:compound 18:sodium-methylpropan-2-olate:t-BuXPhos palladium(II) biphenyl-2-amine mesylate=0.41:1:1.33:0.03, and the mixed solution was filled with nitrogen and reacted at 50° C. for 16 hours and purified to obtain compound 19, 7-methoxy-3-(2H-isoindol-2-yl) isoquinoline: MS (m/z) 275.1 [M+H]⁺.

3. Compound 19 was dissolved in dichloromethane solution, and boron tribromide was added at −78° C., in which molar ratio of compound 19:boron tribromide=1:1, and the mixture being reacted at room temperature for 16 hours and followed by extraction and purification to obtain the compound 20 3-(2H-isoindol-2-yl) isoquinoline-7-ol: MS (m/z) 261.1 [M+H]⁺.

Compound 20 is dissolved in dimethylformamide, and potassium carbonate and 1-bromo-2-p-toluenesulfonyloxymethane were added, in which molar ratio of compound 20:potassium carbonate:1-bromo-2-p-toluenesulfonyloxymethane=1:2.07:1.53, and the mixture after being reacted for 16 hours at 30° C. and followed by extraction and purification to obtain the compound 21 7-(2-p-toluenesulfonyloxymethoxy)-3-(2H-isoindol-2-yl) isoquinoline: MS (m/z) 445.1 [M+H]⁺:

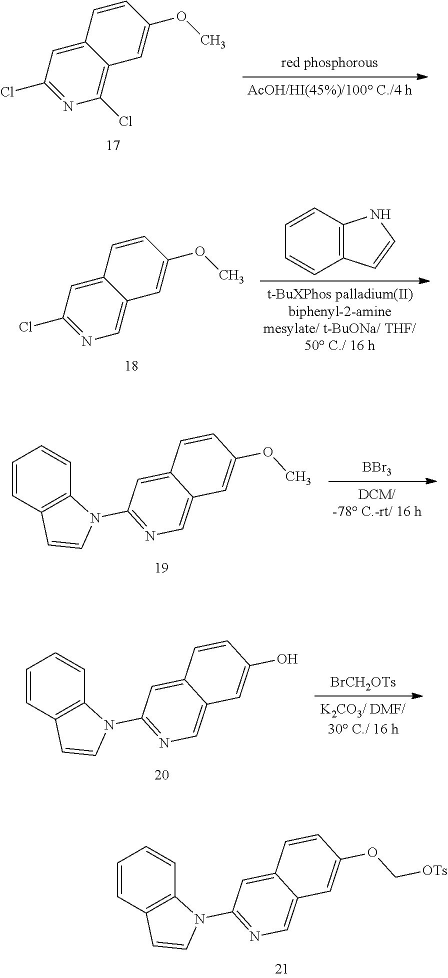

Synthesis of standard 7-(2-fluoromethoxy)-3-(2H-isoindol-2-yl) isoquinoline:

Compound 20 is dissolved in dimethylformamide, and potassium carbonate and 1-bromo-2-fluoromethane were added, in which molar ratio of compound 20:potassium carbonate:1-bromo-2-fluoromethane=1:2.07:1.53, and the mixture being reacted at 30° C. for 16 hours and followed by extraction and purification to obtain the compound 22 7-(2-fluoromethoxy)-3-(2H-isoindol-2-yl) isoquinoline: MS (m/z) 293.1 [M+H]⁺:

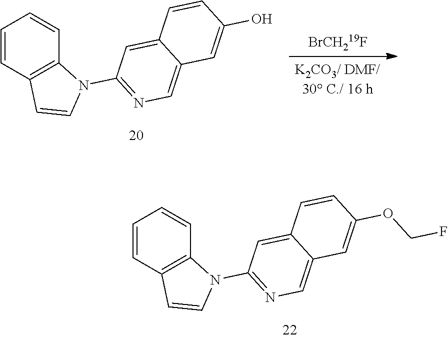 [SMILES: Oc1ccc2cc(-n3ccc4ccccc43)ncc2c1 ; "BrCH219F" ; FCOc1ccc2cc(-n3ccc4ccccc43)ncc2c1]

Synthesis of tracer [18F] 7-(2-fluoromethoxy)-3-(2H-isoindol-2-yl) isoquinoline:

Compound 21 was dissolved in acetonitrile, and potassium carbonate and Kryptofix 222 were added to react at 95° C. for 15 minutes and followed by purification to obtain the compound 23 [18F] 7-(2-fluoromethoxy)-3-(2H-isoindol-2-yl) isoquinoline: MS (m/z) 292.1 [M+H]⁺:

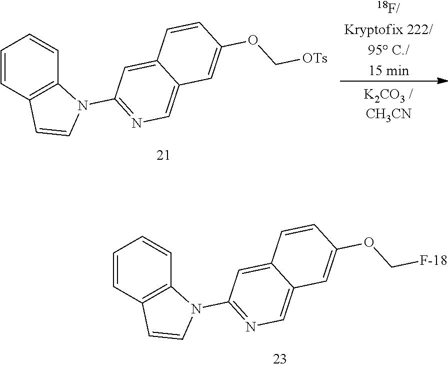

### Embodiment 4

### Synthesis of INER-TAU-R2 Series Compounds

Synthesis of precursor 7-(2-p-toluenesulfonyloxyethoxy)-3-(2H-isoindol-2-yl) isoquinoline, comprising:

1. compound 17, 1,3-dichloro-7-methoxyisoquinoline, is dissolved in a mixed solution of acetic acid and 45% iodine hydride, and red phosphorous is added at room temperature, and the mixed solution is reacted at 100° C. for 4 hours, then cooled to room temperature and purified to obtain compound 18 3-chloro-7-methoxyisoquinoline: MS (m/z) 194.0 [M+H]⁺.

2.3H-Indole and compound 18 were dissolved in tetrahydrofuran, and sodium 2-methylpropan-2-olate and t-BuXPhos palladium(II) biphenyl-2-aminemesylate were added at room temperature, in which molar ratio of 3H-Indole:compound 18:sodium 2-methylpropan-2-olate:t-BuXPhos palladium(II) biphenyl-2-amine mesylate=0.41:1:1.33:0.03, the mixed solution was filled with nitrogen and reacted at 50° C. for 16 hours and purified to obtain compound 19 7-methoxy-3-(2H-isoindol-2-yl) isoquinoline: MS (m/z) 275.1 [M+H]⁺.

3. Compound 19 was dissolved in dichloromethane solution, and boron tribromide was added at −78° C., in which molar ratio of compound 19:boron tribromide=1:1, and then reacted at room temperature for 16 hours and followed by extraction and purification to obtain the compound 20 3-(2H-isoindol-2-yl) isoquinoline-7-ol: MS (m/z) 261.1 [M+H]⁺.

4. Compound 20 was dissolved in dimethylformamide, and potassium carbonate and 1-bromo-2-p-toluenesulfonyloxymethane were added, in which molar ratio of compound 20:potassium carbonate:1-bromo-2-p-toluenesulfonyloxymethane=1:2.07:1.53, and after being reacted at 30° C. for 16 hours and followed by extraction and purification to obtain the compound 24 7-(2-p-toluenesulfonyloxyethoxy)-3-(2H-isoindol-2-yl) isoquinoline: MS (m/z) 459.1 [M+H]⁺:

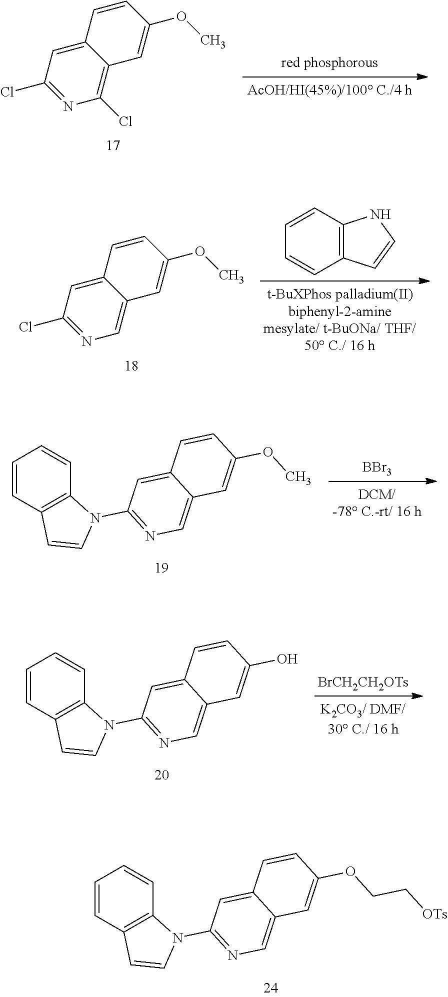

Synthesis of standard 7-(2-fluoroethoxy)-3-(2H-isoindol-2-yl) isoquinoline:

Compound 20 was dissolved in dimethylformamide, and potassium carbonate and 1-bromo-2-fluoromethane were added, in which molar ratio of compound 20:potassium carbonate:1-bromo-2-fluoromethane=1:2.07:1.53, and reacted at 30° C. for 16 hours and followed by extraction and purification to obtain the compound 25, 7-(2-fluoroethoxy)-3-(2H-isoindol-2-yl) isoquinoline: MS (m/z) 307.1 [M+H]⁺:

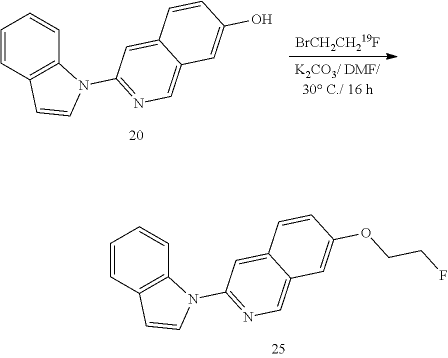

Synthesis of tracer [18F] 7-(2-fluoroethoxy)-3-(2H-isoindol-2-yl) isoquinoline:

Compound 24 was dissolved in acetonitrile, and potassium carbonate and Kryptofix 222 were added to react at 95° C. for 15 minutes and purified to obtain compound 26, [18F] 7-(2-fluoroethoxy)-3-(2H-isoindol-2-yl) isoquinoline: MS (m/z) 306.1 [M+H]⁺:

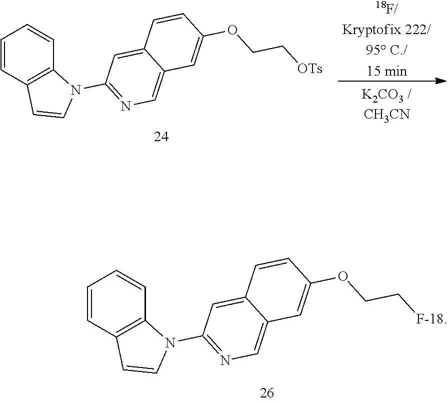

### Embodiment 5

### Synthesis of INER-TAU-R3 Series Compounds

Synthesis of precursor 7-(2-p-toluenesulfonyloxypropoxy)-3-(2H-isoindol-2-yl) isoquinoline, comprising:

1. 1,3-dichloro-7-methoxyisoquinoline compound 17 was dissolved in a mixed solution of acetic acid and 45% iodine hydride, and red phosphorous is added at room temperature, and the mixed solution was reacted at 100° C. for 4 hours, then cooled to room temperature and purified to obtain the compound 18 3-chloro-7-methoxyisoquinoline: MS (m/z) 194.0 [M+H]⁺.

2.3H-Indole and compound 18 were dissolved in tetrahydrofuran, sodium 2-methylpropan-2-olate and t-BuXPhos palladium(II) biphenyl-2-amine mesylate were added at room temperature, in which molar ratio of 3H-Indole:compound 18:sodium 2-methylpropan-2-olate:t-BuXPhos palladium(II) biphenyl-2-amine mesylate=0.41:1:1.33:0.03, and the mixed solution was filled with nitrogen and reacted at 50° C. for 16 hours and purified to obtain compound 19 7-methoxy-3-(2H-isoindol-2-yl) isoquinoline: MS (m/z) 275.1 [M+H]⁺.

3. Compound 19 was dissolved in dichloromethane solution, and boron tribromide was added at −78° C., in which molar ratio of compound 19:boron tribromide=1:1, and then reacted at room temperature for 16 hours and followed by extraction and purification to obtain the compound 20 3-(2H-isoindol-2-yl) isoquinoline-7-ol: MS (m/z) 261.1 [M+H]⁺.

4. Compound 20 was dissolved in dimethylformamide, and potassium carbonate and 1-bromo-2-p-toluenesulfonyloxymethane were added, in which molar ratio of compound 20:potassium carbonate:1-bromo-2-p-toluenesulfonyloxymethane=1:2.07:1.53, and after being reacted at 30° C. for 16 hours and followed by extraction and purification to obtain the compound 27 7-(2-p-toluenesulfonyloxypropoxy)-3-(2H-isoindol-2-yl) isoquinoline: MS (m/z) 473.2 [M+H]⁺:

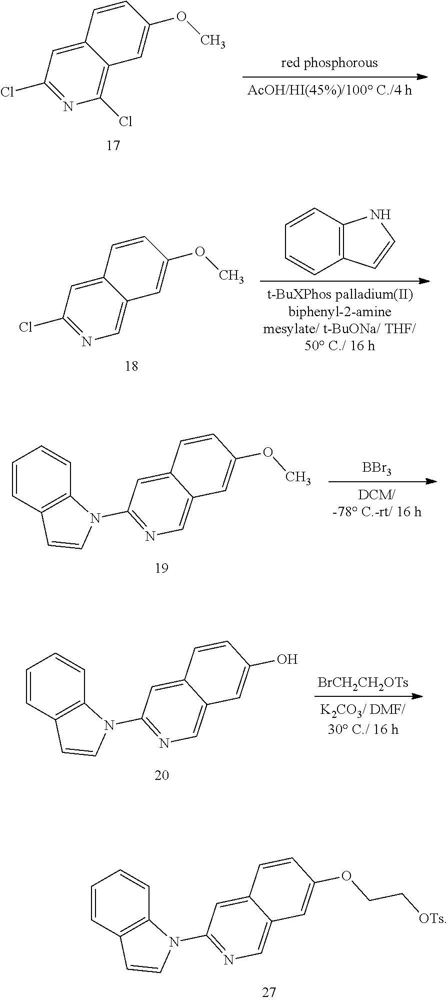

Synthesis of standard 7-(2-fluoropropoxy)-3-(2H-isoindol-2-yl) isoquinoline:

Compound 20 was dissolved in dimethylformamide, and potassium carbonate and 1-bromo-2-fluoromethane, in which molar ratio of compound 20:potassium carbonate:1-bromo-2-fluoropropane=1:2.07:1.53, were added to react at 30° C. for 16 hours and followed by extraction and purification to obtain the compound 28 7-(2-fluoropropoxy)-3-(2H-isoindol-2-yl) isoquinoline: MS (m/z) 321.1 [M+H]⁺:

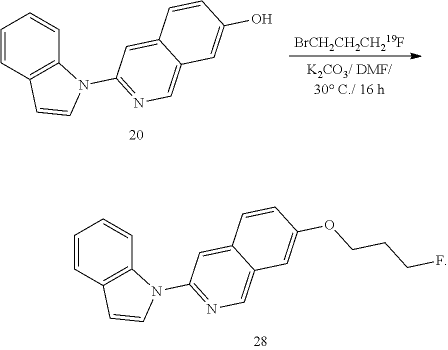

Synthesis of tracer [18F] 7-(2-fluoropropoxy)-3-(2H-isoindol-2-yl) isoquinoline, comprising:

1. Compound 27 was dissolved in acetonitrile, and potassium carbonate and Kryptofix 222 were added to react at 95° C. for 15 minutes and purification to obtain the compound 29 [18F] 7-(2-fluoropropoxy)-3-(2H-isoindol-2-yl) isoquinoline was obtained.: MS (m/z) 320.1 [M+H]⁺;

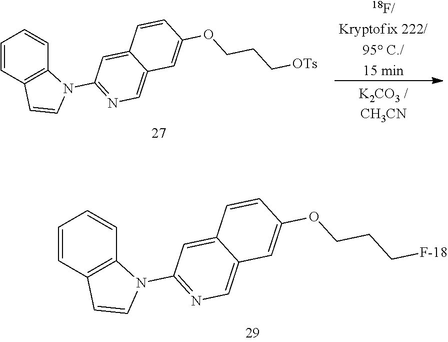

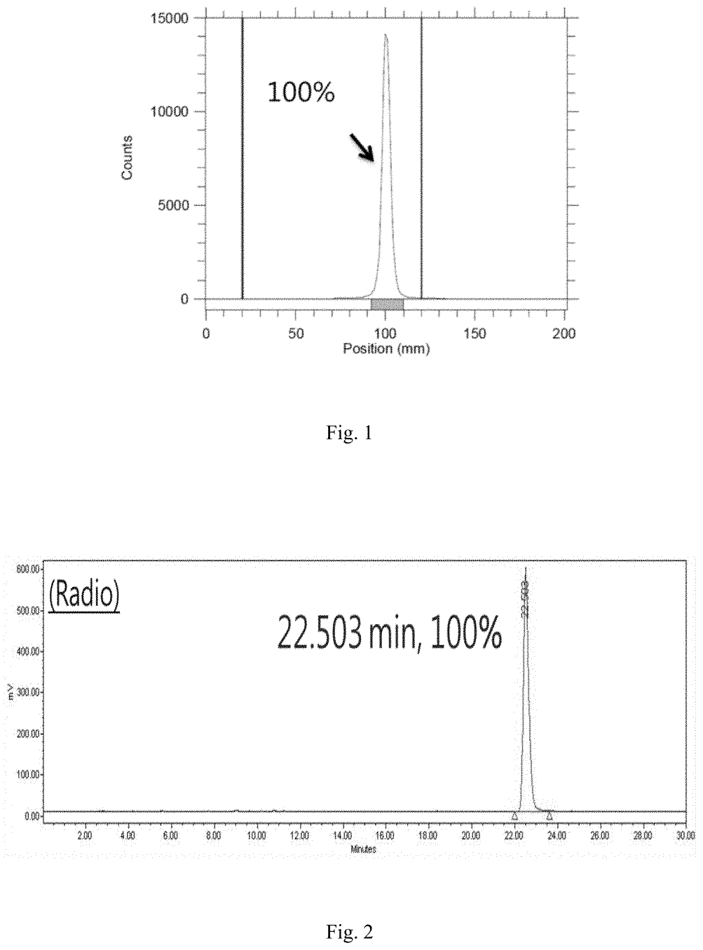

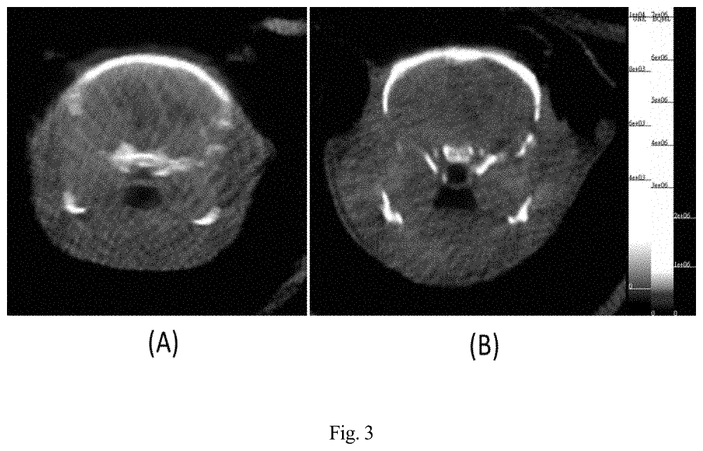

2. The obtained compound 29, [18F] 7-(2-fluoropropoxy)-3-(2H-isoindol-2-yl) isoquinoline, was analyzed by radio-TLC and radio-HPLC to confirm the purity is greater than 95%, as shown in FIG. 1 and FIG. 2.

Radio-TLC analysis uses TLC sheet silica gel 60 F254 (Merck), mobile phase is CH2Cl2: CH3OH=8:2; Radio-HPLC analysis uses XSelect HSS T3 column, mobile phase uses gradient method: from 60% acetonitrile+0.1% TFA to 90% acetonitrile+0.1% TFA in 30 minutes.

0.46 mCi of compound 29 was injected into P301L gene transgenic mice and control mice, and after 30 minutes of distribution nano-PET/CT was used for contrast imaging. The image showed that the absorption of compound 29 in the brain of P301L gene transgenic mice was significantly higher than that of the control mice, as shown in FIG. 3.

In one preferred embodiment of the instant disclosure, there is provided a pharmaceutical composition comprising the pharmaceutical compound or a pharmaceutically acceptable salt thereof as described herein.

In another preferred embodiment of this disclosure, there is provided a method of Tau protein imaging in a biological sample using a pharmaceutical composition described herein, comprising the steps of: (1) providing a cell and/or a tissue and/or an organ sample from a subject, (2) contacting said sample with the pharmaceutical composition, and (3) imaging said sample, wherein deposits of microtubule-associated protein Tau in said sample is detected.

Based on the above, the novel diagnostic microtubule-related protein Tau imaging precursors, standards and tracers provided by the present invention can indeed achieve the characteristics of easy synthesis, high yield and better analytical accuracy. The content of the above description is only a description of the preferred embodiments of the invention, and all the amendments based on the technical means and scope of the invention, including modifications, changes or equivalent substitutions shall also fall within the scope of the claims of the present invention.

What is claimed is:

1. A pharmaceutical compound of formula 1 or a pharmaceutically acceptable salt thereof, 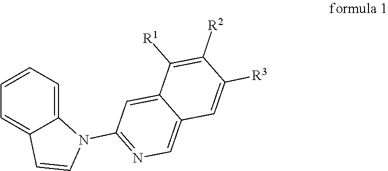
wherein
R1 is a molecule selected from the group consisting of H, I, ¹²³I, ¹²⁴I, ¹²⁵I, and ¹³¹I;
R2 is a molecule selected from the group consisting of H, I, ¹²³I, ¹²⁴I, ¹²⁵I, and ¹³¹I; and
R3 is a molecule selected from the group consisting of H, 2-p-toluenesulfonyloxypropoxyl, 2-fluoropropoxyl, [F-18]2-fluoropropoxyl, 2-p-toluenesulfonyloxyethoxyl, 2-fluoroethoxyl, [F-18]2-fluoroethoxyl, 2-p-toluenesulfonyloxymethoxyl, and 2-fluoromethoxyl, [F-18]2-fluoromethoxyl, wherein at least one of R1, R2 and R3 is not H.

2. The pharmaceutical compound of claim 1 or a pharmaceutically acceptable salt thereof, wherein
R1 is H;
R2 is H; and
R3 is a molecule selected from the group consisting of 2-p-toluenesulfonyloxypropoxyl, 2-fluoropropoxyl, [F-18]2-fluoropropoxyl, 2-p-toluenesulfonyloxyethoxyl, 2-fluoroethoxyl, [F-18]2-fluoroethoxyl, 2-p-toluenesulfonyloxymethoxyl, 2-fluoromethoxyl, and [F-18]2-fluoromethoxyl, wherein the structure of the molecule is shown with the following codes:

| Code | IUPAC | Structure |
|---|---|---|
| INER-TAU- R3-OTs | 7-(2-p- toluenesulfonyloxy- propoxy)-3-(2H- isoindol-2-yl) isoquinoline | 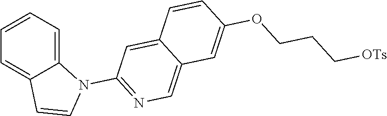 [SMILES: Cc1ccc(S(=O)(=O)OCCCOc2ccc3cc(-n4ccc5ccccc54)ncc3c2)cc1] |
| INER-TAU- R3 | 7-(2-fluoropropoxy)- 3-(2H-isoindol-2-yl) isoquinoline | 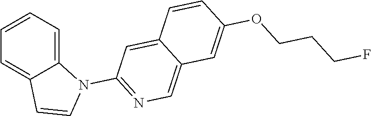 [SMILES: FCCCOc1ccc2cc(-n3ccc4ccccc43)ncc2c1] |
| ¹⁸F-INER- TAU-R3 | [F-18]7-(2- fluoropropoxy)-3- (2H-isoindol-2-yl) isoquinoline | 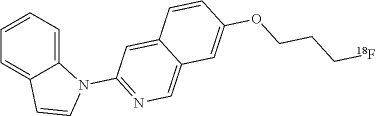 [SMILES: [18F]CCCOc1ccc2cc(-n3ccc4ccccc43)ncc2c1] |
| INER-TAU- R2-OTs | 7-(2-p- toluenesulfonyloxy- ethoxy)-3-(2H- isoindol-2-yl) isoquinoline | 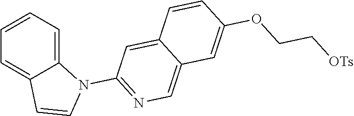 [SMILES: Cc1ccc(S(=O)(=O)OCCOc2ccc3cc(-n4ccc5ccccc54)ncc3c2)cc1] |
| INER-TAU- R2 | 7-(2-fluoroethoxy)-3- (2H-isoindol-2-yl) isoquinoline | 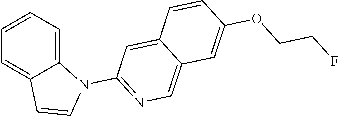 [SMILES: FCCOc1ccc2cc(-n3ccc4ccccc43)ncc2c1] |
| ¹⁸F-INER- TAU-R2 | [F-18]7-(2- fluoroethoxy)-3-(2H- isoindol-2-yl) isoquinoline | 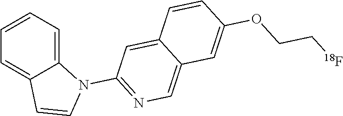 [SMILES: [18F]CCOc1ccc2cc(-n3ccc4ccccc43)ncc2c1] |
| INER-TAU- R1-OTs | 7-(2-p- toluenesulfonyloxy- methoxy)-3-(2H- isoindol-2-yl) isoquinoline | 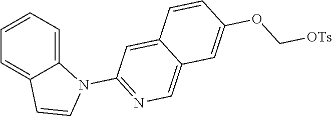 [SMILES: Cc1ccc(S(=O)(=O)OCOc2ccc3cc(-n4ccc5ccccc54)ncc3c2)cc1] |
| INER-TAU- R1 | 7-(2-fluoromethoxy)- 3-(2H-isoindol-2-yl) isoquinoline | 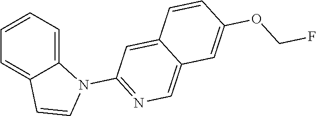 [SMILES: FCOc1ccc2cc(-n3ccc4ccccc43)ncc2c1] |
| ¹⁸F-INER- TAU-R1 | [F-18]7-(2- fluoromethoxy)-3- (2H-isoindol-2-yl) isoquinoline | 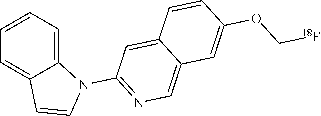 [SMILES: [18F]COc1ccc2cc(-n3ccc4ccccc43)ncc2c1] |

3. The pharmaceutical compound of claim 1 or a pharmaceutically acceptable salt thereof, wherein
R1 is H;
R3 is H; and
R2 is a molecule selected from the group consisting of I, ¹²³I, ¹²⁴I, ¹²⁵I, and ¹³¹I, wherein the structure of the molecule is shown with the following codes:

| Code | IUPAC | Structure |
|---|---|---|
| INER- TAU-I1 | 6-iodo-3-(2H- isoindol-2-yl) isoquinoline | 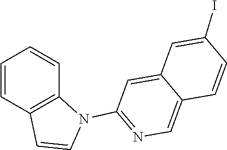 [SMILES: Ic1ccc2cnc(-n3ccc4ccccc43)cc2c1] |
| ¹²³I-INER- TAU-I1 | [¹²³I]6-iodo-3-(2H- isoindol-2-yl) isoquinoline | 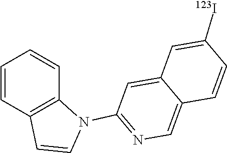 [SMILES: [123I]c1ccc2cnc(-n3ccc4ccccc43)cc2c1] |
| ¹²⁴I-INER- TAU-I1 | [¹²⁴]6-iodo-3-(2H- isoindol-2-yl) isoquinoline | 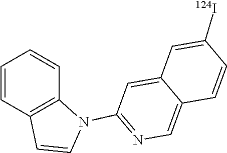 [SMILES: [124I]c1ccc2cnc(-n3ccc4ccccc43)cc2c1] |
| ¹²⁵I-INER- TAU-I1 | [¹²⁵I]6-iodo-3-(2H- isoindol-2-yl) isoquinoline | 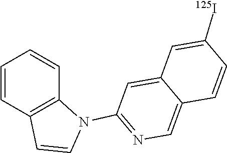 [SMILES: [125I]c1ccc2cnc(-n3ccc4ccccc43)cc2c1] |
| ¹³¹I-INER- TAU-I1 | [¹³¹I]6-iodo-3-(2H- isoindol-2-yl) isoquinoline | 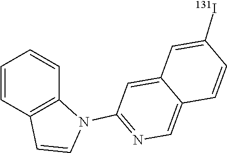 [SMILES: [131I]c1ccc2cnc(-n3ccc4ccccc43)cc2c1] |

4. The pharmaceutical compound of claim 1 or a pharmaceutically acceptable salt thereof, wherein
R2 is H;
R3 is H; and
R1 is a molecule selected from the group consisting of I, ¹²³I, ¹²⁴I, ¹²⁵I, and ¹³¹I, wherein the structure of the molecule is shown with the following codes:

| Code | IUPAC | Structure |
|---|---|---|
| INER- TAU-I2 | 5-iodo-3-(2H- isoindol-2- yl)isoquinoline | 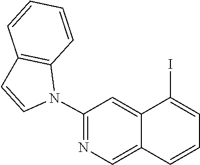 [SMILES: Ic1cccc2cnc(-n3ccc4ccccc43)cc12] |
| ¹²³I-INER- TAU-I2 | [¹²³I]5-iodo-3- (2H-isoindol-2- yl)isoquinoline | 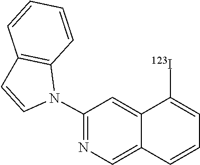 [SMILES: [123I]c1cccc2cnc(-n3ccc4ccccc43)cc12] |
| ¹²⁴I-INER- TAU-I2 | [¹²⁴I]5-iodo-3- (2H-isoindol-2- yl)isoquinoline | 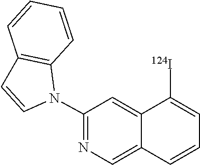 [SMILES: [124I]c1cccc2cnc(-n3ccc4ccccc43)cc12] |
| ¹²⁵I-INER- TAU-I2 | [¹²⁵I]5-iodo-3- (2H-isoindol-2- yl)isoquinoline | 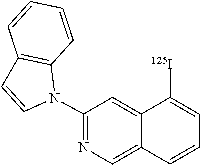 [SMILES: [125I]c1cccc2cnc(-n3ccc4ccccc43)cc12] |
| ¹³¹I-INER- TAU-I2 | [¹³¹I]5-iodo-3- (2H-isoindol-2- yl)isoquinoline | 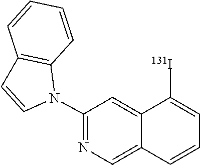 [SMILES: [131I]c1cccc2cnc(-n3ccc4ccccc43)cc12] |

5. A pharmaceutical composition comprising the pharmaceutical compound of claim 1 or a pharmaceutically acceptable salt thereof.

6. A method for producing the pharmaceutical compound of claim 1 or a pharmaceutically acceptable salt thereof, comprising the synthesis steps of:

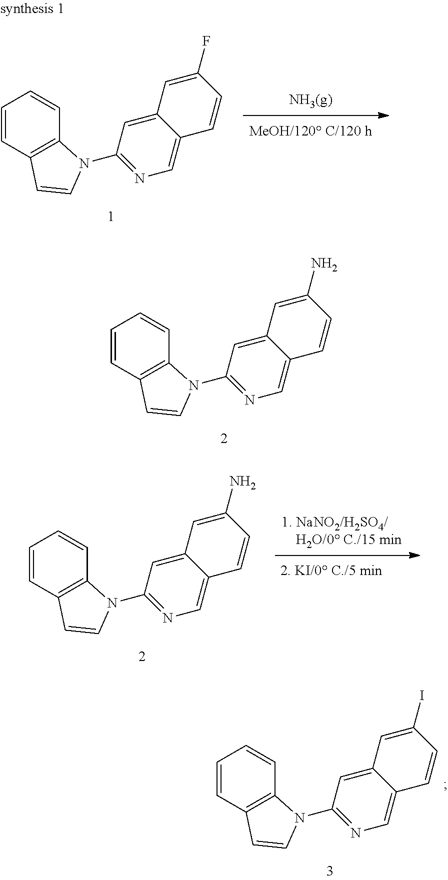 [SMILES: Fc1ccc2cnc(-n3ccc4ccccc43)cc2c1 ; Nc1ccc2cnc(-n3ccc4ccccc43)cc2c1 ; Nc1ccc2cnc(-n3ccc4ccccc43)cc2c1 ; Ic1ccc2cnc(-n3ccc4ccccc43)cc2c1]

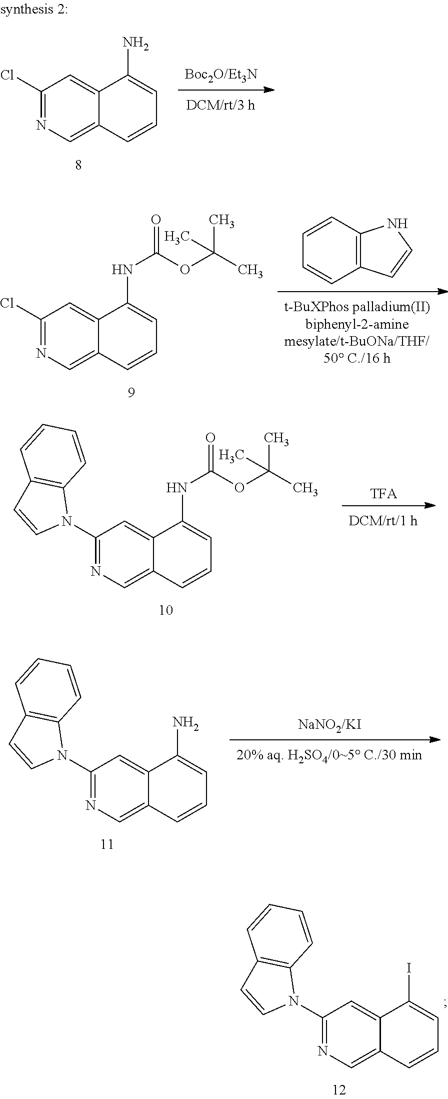

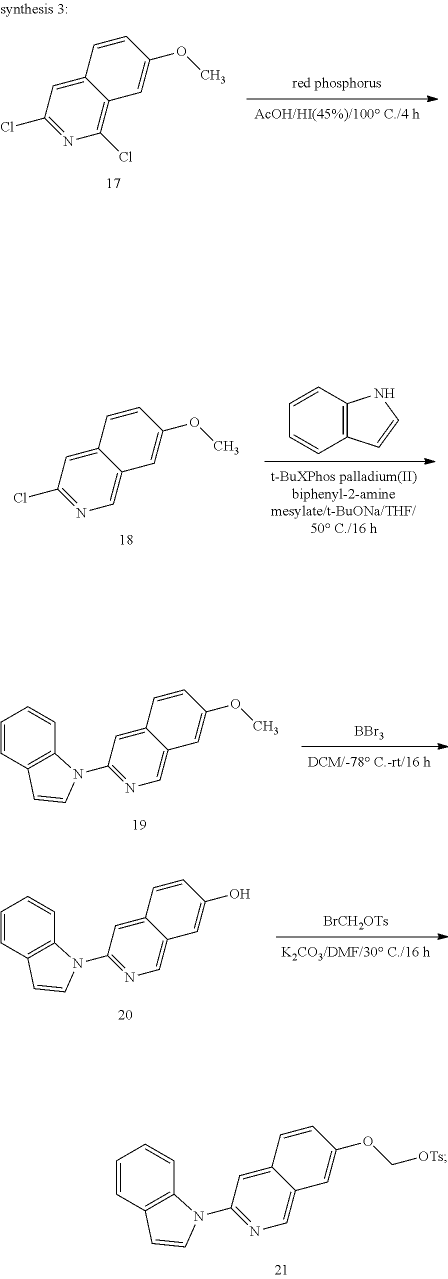

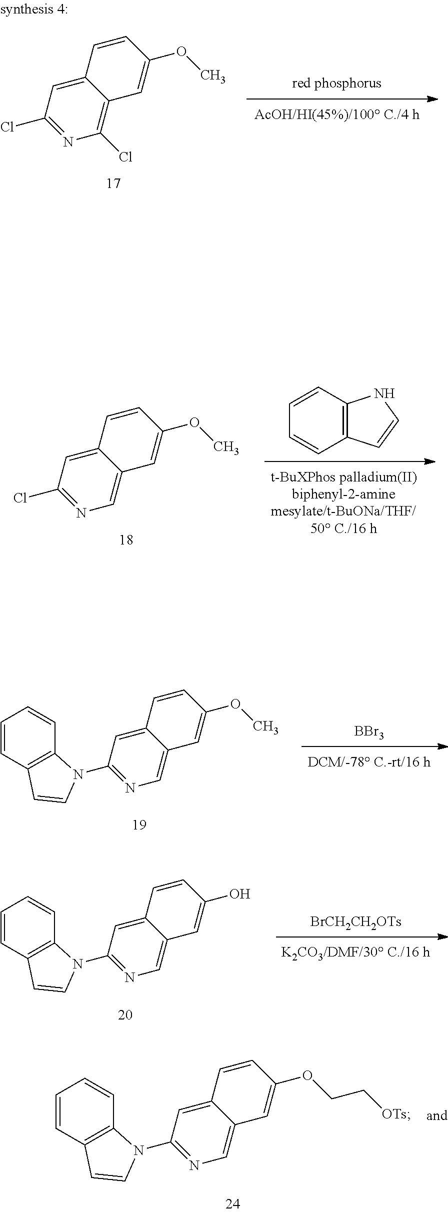

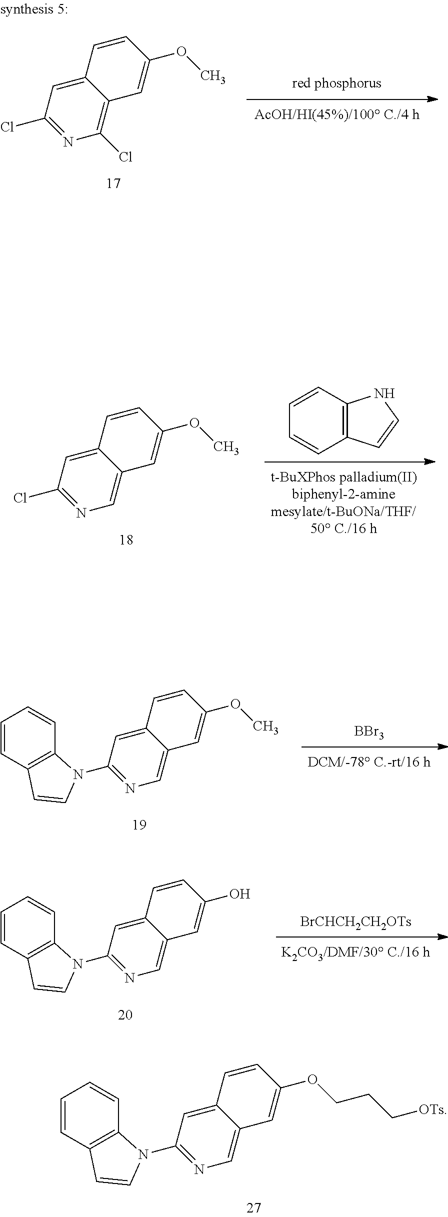

7. A method of Tau protein imaging in a biological sample using the pharmaceutical composition of claim 5, comprising the steps of:
providing a cell and/or a tissue and/or an organ sample from a subject,
contacting said sample with the pharmaceutical composition of claim 5, imaging said sample,
wherein deposits of microtubule-associated protein Tau in said sample is detected.
